(19) United States
(12) Patent Application Publication
(10) Pub. No.: US 2007/0088444 A1
(43) Pub. Date: Apr. 19, 2007

(54) Method for repairing a bone defect using a formable implant which hardens in vivo

(76) Inventors: Robert A Hodorek, Warsaw, IN (US); Brian H Thomas, Columbia City, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/251,181

(22) Filed: Oct. 13, 2005

(51) Int. Cl. A61F 2/28

(52) U.S. Cl. 623/23.61; 424/423

(57) ABSTRACT

A method for the repair of bone defects which requires only the resection of the defective portion of the bone. After resecting a defective portion of the bone, a formable implant may be inserted through an incision in the skin and placed over the resected portion of the bone. The formable implant may conform to the shape of the resected bone, after which the formable implant may be adjusted or formed to a desired shape. Once a desired shape and location are achieved, a catalyst is employed to harden the formable implant.

### BACKGROUND

1. Field of the Invention

The present invention relates to a method for implanting prosthetic implants, and, more particularly, to a method for implanting a formable implant which hardens in vivo.

2. Description of the Prior Art

Many patients experience bone defects which may be caused by a number of factors including age, illness, or trauma. Typically, the bone defects need to be repaired to prevent further decline of the bone structure. Conventional techniques for repair may require the removal of at least some amount of healthy bone surrounding the defective area. For example, during a typical total knee arthroplasty, a surgeon typically must resect an appropriate amount of femoral bone, including healthy portions, to ensure an adequate fit between the distal femur and a distal femoral prosthesis.

What is desired is a technique for repair of diseased bone which is an improvement over the foregoing.

### SUMMARY

The present invention provides a method for the repair of bone defects which requires only the resection of a defective portion of a bone in order to substantially preserve healthy bone stock. After resecting a defective portion of the bone, a formable implant may be inserted through an incision in the skin and placed over or within the resected portion of the bone. The formable implant may conform to the shape of the resected bone portion, after which the formable implant may be adjusted or formed to a desired shape. Once a desired shape and location are achieved, a catalyst is employed to harden the formable implant. Advantageously, the present invention provides a customizable approach to the repair of diseased bone.

In one form thereof, the present invention provides a method for implanting a formable implant to conform to the shape of an anatomical structure including preparing a site on the anatomical structure; shaping the formable implant to substantially match the site on the anatomical structure; delivering the formable implant to the site; shaping an articulating surface on the formable implant; and hardening the formable implant using a catalyst.

In another form thereof, the present invention provides a method for repairing a bone defect associated with a bone including preparing a site on the bone; shaping a formable implant to substantially match the site on the bone; delivering the formable implant to the site; shaping an articulating surface on the formable implant; and hardening the formable implant using a catalyst.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a lateral perspective view of a patient's limb;

FIG. 2 is a perspective view of a femur and a tibia;

FIG. 2A is a fragmentary perspective view of a knee joint showing a resected portion of the distal femur;

FIG. 3A is a fragmentary perspective view of the distal femur of FIG. 2A, with a formable implant shown occupying the resected portion of the distal femur; and

FIG. 3B is a fragmentary perspective view of the distal femur of FIG. 2A, with an alternative formable implant shown occupying the resected portion of the distal femur and extending a distance below the original distal edge of the distal femur.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings.

In general, the present invention provides a method for implanting a formable implant which hardens in vivo. A suitable incision may be made in a patient via a number of techniques well-known in the art. Once the incision is formed, a surgeon can perform a resection of a portion of a bone by any one of a number of well-known techniques. The formable implant may then be inserted via the incision to the site of the resected portion of the bone. The formable implant may be shaped to conform to the resected bone surface either prior to or subsequent to insertion into the patient so as to provide a conforming fit between the formable implant and the bone surface. The surgeon may manipulate and/or trim the formable implant to obtain a desired articulating shape, as necessary. Once the formable implant is correctly positioned and shaped, a catalyst is employed to harden the formable implant.

Although the formable implants disclosed herein are described and illustrated herein in the context of repair of a distal femur in a knee joint, the implants of the present invention may be used elsewhere in a patient such as near a hip joint, a shoulder joint, along a portion of a bone not proximate a joint area, or any other areas of diseased or damaged bone.

Referring now to FIG. 1, limb 10 of a patient is illustrated with incision 12 located proximate knee joint 13. Incision 12 may be formed by any well-known technique and may comprise an incision only a few centimeters long, e.g., 2-5 cm. Incision 12 provides access for the surgeon to perform a resection of a bone surface and to insert formable implant 20, as described hereinbelow.

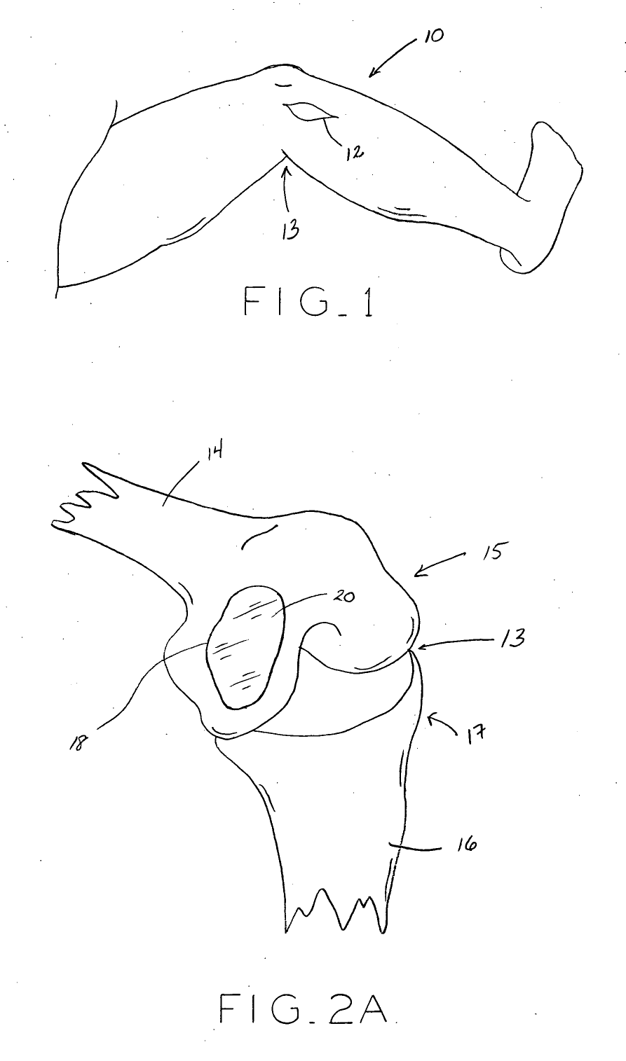

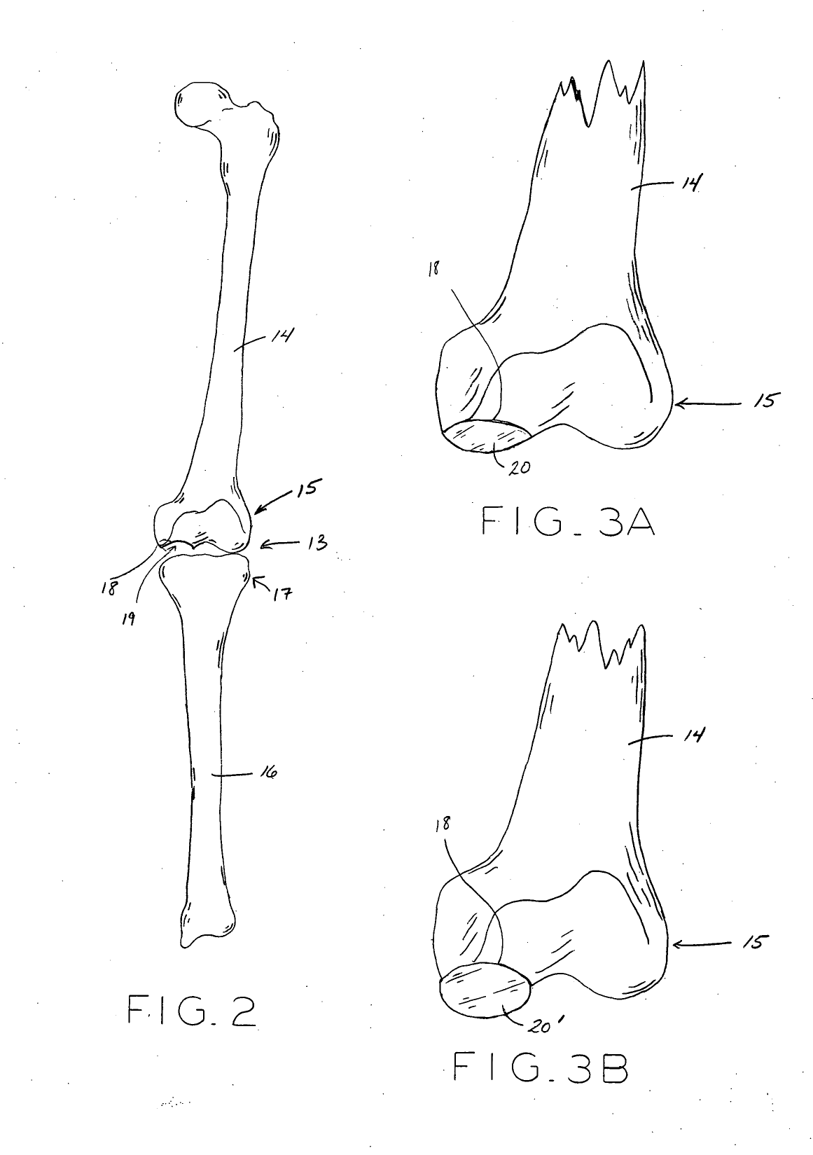

Referring to FIG. 2A, resected site or surface 18 may be formed using any well-known surgical instruments and techniques. Although illustrated in FIG. 2A as encompassing only a portion of the medial condyle of distal femur 15, resected surface 18 may be located on the lateral condyle or both medial and lateral condyles of distal femur 15. Alternatively, resected surface 18 may be located on any portion of proximal tibia 17 of tibia 16 (FIGS. 2 and 2A). Additionally, although described throughout as applied to knee joint 13, resected surface 18 may be formed on any other bone surface having a defective portion and formable implant 20 may be used with any resected bone surface. In one embodiment, resected surface 18 encompasses a defective portion of distal femur 15 and advantageously may be formed to leave substantially intact the remaining healthy bone of femur 14. As shown in FIG. 2, resected surface 18 may be provided at a desired depth into distal femur 15 so as to remove all defective portions from distal femur 15 and create resected cavity 19 while leaving the healthy or undamaged bone stock of distal femur 15 intact.

In one embodiment, resected cavity 19 embodies a removal of bone stock to a depth of between 1 and 10 mm. In an alternative embodiment, resected cavity 19 embodies a removal of bone stock to a depth of between 1 and 4 mm. In a still further embodiment, resected cavity 19 embodies a removal of bone stock to a depth of between 1 and 2 mm. Resected surface 18 could be formed at a depth greater than 10 mm, depending on the desired application.

In one embodiment, resected surface 18 could be located and identified via a computer-assisted surgery (CAS) system. For example, a probe (not shown) may be used to trace out a perimeter around a defective portion of the bone. The probe communicates that information to the CAS system (not shown). The CAS system uses that information to either simulate an appropriate resection cut for distal femur 15 or to provide a plan for resecting distal femur 15. Upon inputting a desired depth based on prior knowledge from imaging scans, e.g., computer tomography (CT) imaging, magnetic resonance imaging (MRI), fluoroscopic imaging, etc., of distal femur 15, the CAS system may provide plans or simulations of the removal of defective bone to a certain depth. Furthermore, the CAS system may also provide plans or simulations for the implantation process of formable implant 20.

Referring now to FIGS. 2A and 3A, formable implant 20 may be inserted via incision 12 into limb 10, as described below, and positioned on resected surface 18 to occupy resected cavity 19. In one embodiment, formable implant 20 completely occupies resected cavity 19 and provides an identical shape to the original bone structure of distal femur 15, as shown in FIG. 3A. Formable implant 20′ is shown in FIG. 3B which, except as described below, is substantially similar in structure and operation to formable implant 20 (FIGS. 2A and 3A) described herein. As shown in FIG. 3B, formable implant 20′ provides a shape different than that of the original bone structure of distal femur 15 by providing a portion thereof extending distally from distal femur 15. The portion of formable implant 20′ extending from distal femur 15 may advantageously be employed to correct for varus deformity of knee joint 13, for example. Alternatively, formable implant 20′ may be positioned on the lateral condyle (not shown) to correct for valgus deformity of knee joint 13, for example.

Once formable implant 20 is positioned on resected surface 18, formable implant 20 may be manipulated and shaped to conform formable implant 20 to the shape of the bone of resected surface 18. For example, a surgeon may press formable implant 20 onto resected surface 18 to ensure adequate contact between formable implant 20 and resected surface 18. Pressing or applying formable implant 20 onto resected surface 18 shapes the bone-contacting surface of formable implant 20 to match the bone surface of resected surface 18. Formable implant 20 may also be manipulated or shaped so as to provide a suitable articulating surface on the portion facing away from resected surface 18. The articulating surface would, in one embodiment, have a very smooth and lubricious surface with a low coefficient of friction. A surgeon may use any instrument suitable for manipulation of formable implant 20 to provide the suitable articulating surface and to ensure that formable implant 20 fully contacts resected surface 18. After conforming and shaping formable implant 20, formable implant 20 is hardened via a catalyst, as described below. The hardening of formable implant 20 provides a solid articulating portion of distal femur 15 to cooperate with proximal tibia 17 in knee joint 13.

Formable implant 20 may be constructed in several different ways. In one embodiment, formable implant 20 may be a woven construct which may include a fabric material or a plurality of fibers. The woven construct may be formed to have a thickness to provide formable implant 20 with some depth, depending on the desired application or depth of resected cavity 19. In one embodiment, the woven construct would remain flexible to allow ease of insertion and to facilitate conforming formable implant 20 to resected surface 18. The woven construct may be formed of fibers constructed from metals, including titanium, metal alloys, cobalt-chrome, or other materials such as polymers, fabrics, plastics, or other biocompatible materials, e.g., polyetheretherketone (PEEK), silicon, or polymethylmethacrylate (PMMA). Additionally, the woven construct may be formed of bioresorbable materials which, over time, resorb into the body and allow bone stock to grow into the voids created as the material resorbs.

In one embodiment, formable implant 20 could be constructed in a variety of pre-formed shapes advantageously removing the need to trim or cut formable implant 20 intraoperatively. In this manner, the surgeon could have templates that matched the pre-formed shapes and the surgeon could place the template against the defective portion of the bone, whereby the surgeon would choose the correct size implant to completely cover the defective portion. The surgeon could mark on the bone the boundaries of the resection and then prepare the bone within that template so that formable implant 20 substantially covers resected surface 18. In an alternative embodiment, formable implant 20 may be cut or trimmed to size intraoperatively either before or after insertion without the use of any pre-formed shape or templates.

A portion of the surface of formable implant 20 contacting resected surface 18 may contain an attachment facilitator which helps to attach formable implant 20 to distal femur 15. In one embodiment, fibrin glue, i.e., a commercially available bio-glue, may be used between formable implant 20 and resected surface 18. In another embodiment, formable implant 20 may include a plastic or metal mesh material on the surface contacting resected surface 18 to facilitate the ingrowth of bone into formable implant 20 after implantation in knee joint 13. In one embodiment, formable implant 20 may be formed of a highly porous biomaterial useful as a bone substitute and/or cell and tissue receptive material. An example of such a material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, the disclosure of which is incorporated herein by reference. As would be apparent to one skilled in the art, although the embodiments described herein utilize porous tantalum, other metals such as niobium, or alloys of tantalum and niobium with one another or with other metals may also be used.

In one embodiment, formable implant 20 may be formed entirely of permanent, i.e., non-bioresorbable, material. In another embodiment, formable implant 20 may be formed at least in part of permanent material and at least in part of bioresorbable material. The bioresorbable material permits, over time, for the fibrous tissue of natural bone to interdigitate into formable implant 20 to provide stronger fixation of formable implant 20 to distal femur 15. In yet another embodiment, formable implant 20 may be formed entirely of bioresorbable material, wherein formable implant 20 may include growth factors and stimulus to promote the ingrowth of bone into formable implant 20. Bioresorbable materials suitable for use as formable implant 20 include zoledronate/zoledronic acid (1-hydroxy-2-[(1H-imidazol-1-yl)ethylidine]-bisphosphonic acid); pamidronate (3-amino-1-hydroxypropylidene bisphosphonic acid); alendronate (4-amino-1-hydroxybutylidene bisphosphonic acid); etidronate (1-hydroxyethylidene bisphosphonic acid); clodronate (dichloromethylene bisphosphonic acid); risedronate (2-(3-pyridinyl)-1-hydroxyethylidene bisphosphonic acid); tiludronate (chloro-4-phenylthiomethylidene bisphosphonic acid); ibandronate (1-hydroxy-3(methylpentylamino)-propylidene bisphosphonic acid); incadronate: (cycloheptyl-amino-methylene bisphosphonic acid); minodronate:([1-hydroxy-2-(imidazo[1,2-a]pyridin-3-yl)ethylidene]bi-sphosphonic acid); olpadronate: ((3-dimethylamino-1-hydroxypropylidene) bisphosphonic acid); neridronate (6-amino-1-hydroxyhexylidene-1,1-bisphosphonic acid); EB-1053:1-hydroxy-3-(1-pyrrolidinyl)-propylidene-1,1-bisphosphonic acid; or any other therapeutically effective bisphosphonate or pharmaceutically acceptable salts or esters thereof. The bioresorbable materials used in formable implant 20 may be used in combination with calcium phosphate compounds such as hydroxyapatite.

In one embodiment, the insertion of formable implant 20 into limb 10 may be accomplished by rolling up formable implant 20 and inserting formable implant 20 through a small incision, such as incision 12. In this manner, incision 12 does not need to be very large. The flexibility of formable implant 20 advantageously facilitates such an insertion whereas if formable implant 20 were non-flexible, or rigid, before insertion, a larger incision would be required for insertion. Formable implant 20 may be manipulated inside limb 10 via arthroscopic equipment to conform to resected surface 18 and to shape the articulating surface of formable implant 20, as described above. In an alternative embodiment, formable implant 20 may be folded for insertion through incision 12, and similarly manipulated inside limb 10 via arthroscopic equipment.

The methods of hardening formable implant 20 via a catalyst will now be described. In one embodiment, formable implant 20 may be hardened via a catalyst such as ultraviolet (UV) light. In such an embodiment, formable implant 20 may be formed of material which is flexible and pliable until exposed to UV light, at which point the material hardens into a solid implant. The UV-light curing of materials is a photochemical polymerization process which can be performed on several different materials, such as monomers and ceramics, which polymerize or cross-link (harden or cure) upon exposure to UV light radiation. The different materials used may vary and are essentially composed of base polymers, non-solvent diluents and photo initiators.

In an alternative embodiment, formable implant 20 may be a woven three-dimensional construct comprised of a plurality of hydrogel fibers. In such an embodiment, the catalyst may comprise an aqueous solution containing, for example, water. Hydrogel expands when it absorbs water. Prior to implantation, the hydrogel fibers are in a dry condition and therefore allow formable implant 20 to be pliable and flexible. Once implanted, conformed, and shaped inside limb 10, the aqueous solution may be introduced proximate formable implant 20, thereby causing the hydrogel fibers to expand and interlock formable implant 20 into a rigid structure. The hydrogel fibers may be produced using polymer material such as polyacrylates (e.g. polymethacrylate, polyhydroxyethylmethacrylate (polyHEMA), and polyhydroxypropylmethacrylate), polyvinylpyrollidone (PVP), polyvinyl alcohol (PVA), polyacrylamides, polyacrylonitriles, polysaccharides (e.g. carrageenans and hyaluronic acid), polyalginates, polyethylene oxides (e.g. polyethylene glycol (PEG) and polyoxyethylene), polyamines (e.g. chitosan), polyurethanes (e.g. diethylene glycol and polyoxyalkylene diols), and polymers of ring-opened cyclic esters. The polymers may be crosslinked by the use of photocuring, which employs radiation using UV, X- or Gamma rays to create links or bonds between the polymers. The polymers may alternatively be crosslinked by exposing the polymers to a crosslinking agent, for example, aqueous ion solutions. Other suitable crosslinking agents may include dimethyl aniline, dimethylaminoethyl acetate, sodium thiosulfate, methylene bis-acrylamide, and diisothiocyanate.

In one embodiment, the hydrogel fiber construct may also act as a delivery vehicle for delivering pharmaceuticals and therapeutics to resected surface 18. The hydrogel construct may contain pharmaceuticals such as antibiotics, steroids, anticoagulants, and anti-inflammatories. The hydrogel construct may also include therapeutics including growth factors, tissue response modifiers, nucleic acids/proteins, cytokines, antibodies, blood, periosteal cells (cells of the fibrous membrane covering bone), precursor tissue cells, chondrocytes, fibrocytes, and stem cells. These pharmaceuticals and therapeutics can be used to promote tissue and bone growth, promote endothelialisation, prevent fibrinosis, and fight infection. In an alternative embodiment, the hydrogel fibers may be bioresorbable and, thus, may gradually dissolve as the tissue is rebuilt.

In a still further embodiment, formable implant 20 may comprise a fluidized mixture of a biocompatible polymer, e.g., a silicone or polyurethane polymer, and a biocompatible hydrogel. After implanting the fluidized mixture, the polymer and hydrogel mixture can be solidified by means such as ultraviolet radiation, which can be introduced into the subcutaneous area by a fiber optic device.

In yet another alternative embodiment, formable implant 20 may be hardened via a chemical reaction. For example, formable implant 20 may be formed of material which is pliable and flexible in a given state, but when mixed with another chemical, the entire material hardens to form a solid structure. In one embodiment, formable implant 20 may be formed of a two-part epoxy composition wherein a base compound has a hardener applied to it immediately prior to insertion through incision 12. In this embodiment, formable implant 20 would remain pliable long enough for the surgeon to conform and shape formable implant 20 to resected surface 18 as well as shape the articulating surface of formable implant 20 to a desired shape, after which formable implant 20 would eventually become rigid. In this embodiment, formable implant 20 may be constructed with fibers coated with an epoxy coating. Formable implant 20 may first be placed onto resected surface 18 after which a chemical catalyst, such as amine, would be applied to formable implant 20. The interaction between formable implant 20 and the amine would cause formable implant 20 to harden and maintain the shape of formable implant 20.

In an alternative embodiment, formable implant 20 may be a woven construct in which some of the fibers have an epoxy coating, some of the fibers have an amine coating, and all of the fibers have a protective coating. The fibers are woven such that the fibers with an epoxy coating alternate with the fibers having an amine coating. The protective coating on all the fibers, or, alternatively, at least on all the epoxy-coated fibers or on all the amine-coated fibers, prevents the epoxy from reacting with the amine earlier than desired. Formable implant 20 may be placed onto resected surface 18 and manipulated to form the correct shape and articulation, after which a solution, e.g., an aqueous solution, may be added to formable implant 20 which dissolves the protective coating. The epoxy can then interact with the amine and harden and maintain the shape of formable implant 20.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for implanting a formable implant to conform to the shape of an anatomical structure, comprising:
preparing a site on the anatomical structure;
shaping the formable implant to substantially match the site on the anatomical structure;
delivering the formable implant to the site;
shaping an articulating surface on the formable implant; and
hardening the formable implant.

2. The method of claim 1, further comprising the additional step of trimming the formable implant prior to or subsequent to said hardening step.

3. The method of claim 1, wherein said preparing step comprises resecting at least a defective portion of the anatomical structure.

4. The method of claim 3, wherein said resecting step comprises resecting a portion of the anatomical structure to a depth between 1 and 10 mm.

5. The method of claim 1, wherein said hardening step comprises curing the formable implant with a radiation source.

6. The method of claim 1, wherein the formable implant comprises, at least in part, hydrogel fibers, and said hardening step comprises applying an aqueous solution to the formable implant.

7. The method of claim 1, wherein the formable implant comprises a composition having a first part and a second part, and said hardening step comprises adding said second part to said first part.

8. The method of claim 7, wherein said first part comprises, at least in part, epoxy, and said second part comprises, at least in part, amine.

9. The method of claim 1, wherein said delivery step precedes said first shaping step.

10. The method of claim 1, wherein the formable implant comprises a composition having a first part and a second part, at least one of said first part and said second part including a protective coating, and said hardening step comprises adding an aqueous solution to the formable implant to dissolve said protective coating.

11. The method of claim 1, wherein said hardening step utilizes a catalyst, a photoinitiator, a thermal initiator, metal alkoxides, or a covalent bond-forming reaction.

12. The method of claim 1, wherein the formable implant comprises, at least in part, an element selected from the group consisting of an acrylate, a methacrylate, a vinyl group, a biodegradable material, antibiotics, analgesics, growth factors, hydroxyapatite, osteochondral cells, stem cells, radio-opacifiers, and osteoconductive material.

13. A method for repairing a bone defect associated with a bone, comprising:
preparing a site on the bone;
shaping a formable implant to substantially match the site on the bone;
delivering the formable implant to the site;
shaping an articulating surface on the formable implant; and
hardening the formable implant.

14. The method of claim 13, further comprising the additional step of trimming the formable implant prior to or subsequent to said hardening step.

15. The method of claim 13, wherein said preparing step comprises resecting at least a defective portion of the bone.

16. The method of claim 15, wherein said resecting step comprises resecting a portion of the bone to a depth between 1 and 10 mm.

17. The method of claim 13, wherein said hardening step comprises curing the formable implant with a radiation source.

18. The method of claim 13, wherein the formable implant comprises, at least in part, hydrogel fibers, and said hardening step comprises applying an aqueous solution to the formable implant.

19. The method of claim 13, wherein the formable implant comprises a composition having a first part and a second part, and said hardening step comprises adding said second part to said first part.

20. The method of claim 19, wherein said first part comprises, at least in part, epoxy, and said second part comprises, at least in part, amine.

21. The method of claim 13, wherein said delivery step precedes said first shaping step.

22. The method of claim 13, wherein the formable implant comprises a composition having a first part and a second part, at least one of said first part and said second part including a protective coating, and said hardening step comprises adding an aqueous solution to the formable implant to dissolve said protective coating.

23. The method of claim 13, wherein said hardening step utilizes a catalyst, a photoinitiator, a thermal initiator, metal alkoxides, or a covalent bond-forming reaction.

24. The method of claim 13, wherein the formable implant comprises, at least in part, an element selected from the group consisting of an acrylate, a methacrylate, a vinyl group, a biodegradable material, antibiotics, analgesics, growth factors, hydroxyapatite, osteochondral cells, stem cells, radio-opacifiers, and osteoconductive material.
